(19) United States
(12) Patent Application Publication
(10) Pub. No.: US 2006/0130209 A1
(43) Pub. Date: Jun. 22, 2006

(54) Garment for patients in endoscopy

(75) Inventor: Salman Golan, Tirat Carmel (IL)

Correspondence Address: BRUCE E. LILLING;LILLING & LILLING P.C., P.O. BOX 560, GOLDEN BRIDGE, NY, 10526, US

(73) Assignee: Sightline Technologies Ltd., Matam, Haifa (IL)

(21) Appl. No.: 10/559,300

(22) PCT Filed: May 5, 2004
(86) PCT No.: PCT/IL04/00372

§ 371 Date: Dec. 1, 2005

(30) Foreign Application Priority Data
Jun. 10, 2003 (IL) 156381

(51) Int. Cl. A41D 10/00

(52) U.S. Cl. 2/114

(57) ABSTRACT

A garment (20) to be worn by a patient (22) during endoscopy is cut and sized to fit snugly around a pelvic area of the patient, such that when the garment (20) is worn by the patient (22), the fabric defines two leg holes and an aperture (28), which is positioned adjacent to a body orifice of the patient (22) so as to permit insertion of an endoscope (40) through the aperture (28) into the body orifice.

### FIELD OF THE INVENTION

The present invention relates generally to garments for use in medical procedures, and specifically to garments to be worn by patients during endoscopy.

### BACKGROUND OF THE INVENTION

A patient undergoing endoscopic examination of the rectum and/or colon must generally lie on an examination table with the lower part of his or her body bared, or at best partly covered by a loose drape. Needless to say, this situation is uncomfortable and embarrassing for the patient. In addition, fluids commonly leak out through the patient's anus during the examination, wetting the patient's body and the examining table.

### SUMMARY OF THE INVENTION

Embodiments of the present invention provide a novel garment to be worn by patients undergoing endoscopic examination. The garment is typically made of absorbent fabric, which is shaped to fit over the pelvic area of the patient in the manner of short pants or a diaper. An aperture is cut through the garment in the area of a body orifice, such as the anus, vagina or urethra, through which the endoscope is to be inserted into the patient's body. The aperture permits access to the orifice for insertion of the endoscope therethrough, while the patient is wearing the garment. Thus, the patient's modesty and comfort are protected during the examination. Fluids that leak out of the orifice are typically absorbed by the garment.

In some embodiments of the present invention, an endoscope retainer is fixed to the garment in the area of the aperture. The physician inserts the endoscope through the retainer into the patient's body orifice. As the physician advances the endoscope into the body, the retainer holds the endoscope in position. Thus, the physician may, when necessary, remove both hands from the endoscope to perform other operations, without requiring an assistant to hold the endoscope in the meanwhile.

There is therefore provided, in accordance with an embodiment of the present invention, an endoscopy system including:

an endoscope adapted to be inserted into a body orifice of a patient; and

an endoscope-ready garment having an aperture sized and located to permit insertion of the endoscope into the body orifice of the patient while the garment is worn by the patient.

In a disclosed embodiment, the garment includes a sheet of material, having front and rear portions joined by a central portion, which is cut to fit between the legs, and one or more fasteners, which are adapted to fix the front and rear portions together so as to cover the pelvic area.

In an aspect of the invention, the system includes an endoscope retaining device, which is fastened within the aperture so as to hold the endoscope in place when the endoscope is inserted through the body orifice. Typically, the endoscope retaining device includes a rigid material having a bore therethrough, wherein the bore is sized to fit snugly around the endoscope, and wherein the garment includes a fastening assembly for fixing the rigid material within the third hole.

In a disclosed embodiment, the endoscope is adapted to be inserted into an anus of the patient, and the aperture in the garment is sized and located to permit insertion of the endoscope into the anus.

There is also provided, in accordance with an embodiment of the present invention, an endoscopy system including:

an endoscope-adapted garment having an aperture sized and located to permit insertion of an endoscope therethrough into a body orifice of a patient while the garment is worn by the patient; and

an endoscope extending through the orifice in the garment.

There is additionally provided, in accordance with an embodiment of the present invention, a garment to be worn by a patient during endoscopy, the garment including a fabric, which is cut and sized to fit snugly around a pelvic area of the patient, such that when the garment is worn by the patient, the fabric defines two leg holes and an aperture, which is positioned adjacent to a body orifice of the patient so as to permit insertion of an endoscope through the aperture into the body orifice.

There is further provided, in accordance with an embodiment of the present invention, apparatus for endoscopy, including:

an endoscope-adapted garment having an aperture sized and located to permit insertion of an endoscope therethrough into a body orifice of a patient while the garment is worn by the patient; and

an endoscope retaining device, which is fixed within the aperture so as to hold the endoscope in place when the endoscope is inserted into the body orifice.

There is moreover provided, in accordance with an embodiment of the present invention, a method for endoscopic examination of a patient, including:

fitting a garment around a pelvic area of the patient, the garment having an aperture sized and located to permit insertion of an endoscope therethrough into a body orifice of a patient while the garment is worn by the patient; and

inserting an endoscope through the aperture into the body orifice.

The present invention will be more fully understood from the following detailed description of the embodiments thereof, taken together with the drawings in which:

### BRIEF DESCRIPTION OF THE DRAWINGS

FIGS. 1A and 1B are schematic, pictorial, front and rear illustrations of a garment worn by a patient during endoscopy, in accordance with an embodiment of the present invention; and

FIG. 2 is a schematic top view of a fabric pattern used in producing the garment of FIGS. 1A and 1B.

### DETAILED DESCRIPTION OF EMBODIMENTS

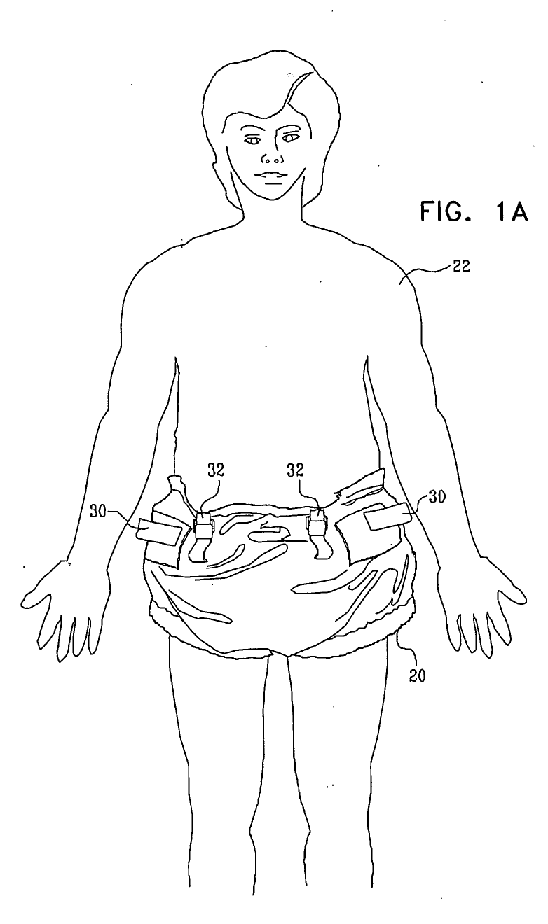

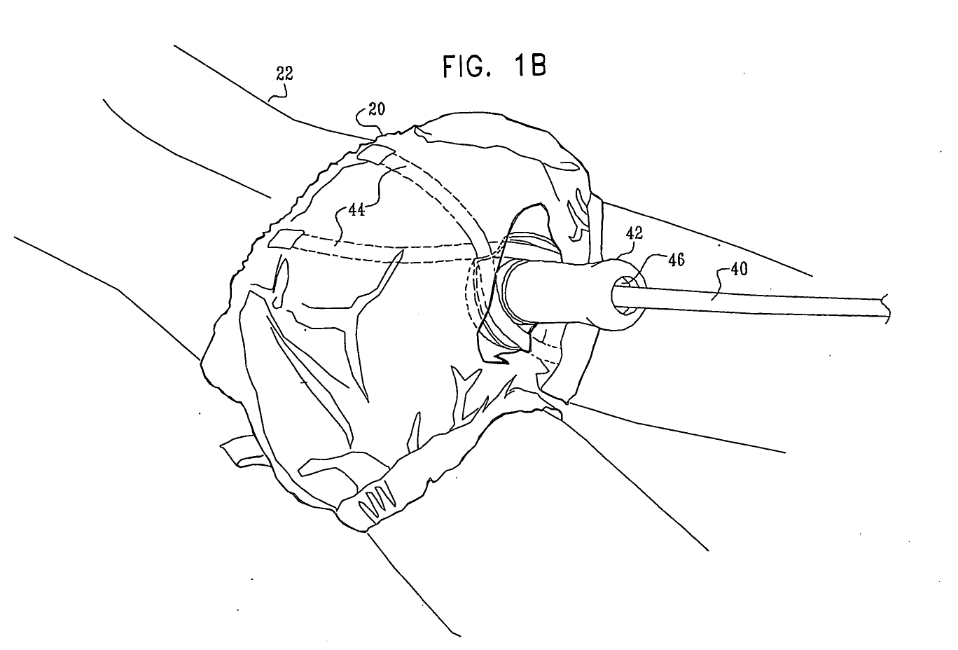

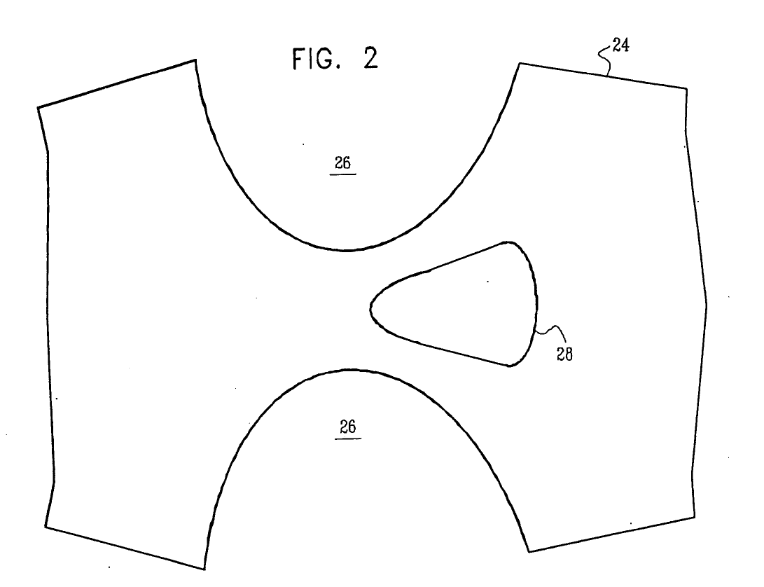

Reference is now made to FIGS. 1A, 1B and 2, which schematically illustrate a garment 20 worn by a patient 22 during endoscopy, in accordance with an embodiment of the present invention. FIGS. 1A and 1B are pictorial front and rear views, respectively. FIG. 2 is a schematic top view of a fabric pattern 24 used in producing garment 20. The pattern has the general form of a diaper, with leg openings 26 on either side of the central portion of the fabric, and (unlike a conventional diaper) an anal aperture 28. The fabric used to produce garment 20 typically comprises absorbent cloth or paper, possibly with an outer waterproof coating, in order to absorb any fluids that leak out of the patient's anus during the endoscopic examination, and to prevent the fluids from wetting the examining table and other areas of the patient's skin or clothing. Fasteners 30, typically Velcro™ or adhesive fasteners, are used to fix the front and rear portions of garment 20 together around the patient's pelvic region. Straps 32 may be used to adjust the garment for a snug fit.

Alternatively, garment 20 may have the form of short pants, rather than a diaper, with elastic to hold the pants around the patient's waist and thighs.

As shown in FIG. 1B, during the endoscopic examination, the physician inserts an endoscope 40, such as a colonoscope, through aperture 28 into the anus of patient 22 optionally, the endoscope is inserted through an endoscope retainer 42, which is held in place adjacent to the anus. In the pictured embodiment, retainer 42 is held by a fastening assembly, such as a system of straps 44, which are provided for this purpose as a part of garment 20. Alternatively, retainer 42 may be sewn, glued, clipped or fastened to garment 20 by any other suitable means known in the art. Still further alternatively, aperture 28 may be made sufficiently small and stiff to serve on its own as the endoscope retaining device, without the use of an additional retaining element such as retainer 42.

Retainer 42 typically comprises a rigid plastic material, with a central bore 46 that is preferably sized to fit snugly around endoscope 40. The fit is loose enough so that the physician can insert and withdraw endoscope 40 through bore 46 without substantial resistance, but still tight enough so that when the physician releases his grip on endoscope 40, the endoscope stays in place and does not begin to slide outward. Retainer 42 thus obviates the need for a nurse or other assistant to hold the endoscope while the doctor performs other operations, such as a biopsy.

After the endoscopic examination is finished, garment 20 and retainer 42 are typically removed and discarded.

Although the embodiment described above relates specifically to endoscopic examination of the rectal area and colon, garment 20 may similarly be adapted for use in endoscopic examination made through other body passages, such as the vagina and urethra. It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. An endoscopy system comprising:
an endoscope adapted to be inserted into a body orifice of a patient; and
an endoscope-ready garment having an aperture sized and located to permit insertion of the endoscope into the body orifice of the patient while the garment is worn by the patient.

2. The system according to claim 1, wherein the garment comprises:
a sheet of material, having front and rear portions joined by a central portion, which is cut to fit between the legs; and
one or more fasteners, which are adapted to fix the front and rear portions together so as to cover the pelvic area.

3. The system according to claim 1, and comprising an endoscope retaining device, which is fastened within the aperture so as to hold the endoscope in place when the endoscope is inserted through the body orifice.

4. The system according to claim 3, wherein the endoscope retaining device comprises a rigid material having a bore therethrough, wherein the bore is sized to fit snugly around the endoscope, and wherein the garment comprises a fastening assembly for fixing the rigid material within the third hole.

5. The system according to claim 1, wherein the endoscope is adapted to be inserted into an anus of the patient, and wherein the aperture in the garment is sized and located to permit insertion of the endoscope into the anus.

6. An endoscopy system comprising:
an endoscope-adapted garment having an aperture sized and located to permit insertion of an endoscope therethrough into a body orifice of a patient while the garment is worn by the patient; and
an endoscope extending through the orifice in the garment.

7. The system according to claim 6, wherein the aperture in the garment is sized and located to permit insertion of the endoscope into an anus of the patient, and wherein the endoscope extends through the anus.

8. A garment to be worn by a patient during endoscopy, the garment comprising a fabric, which is cut and sized to fit snugly around a pelvic area of the patient, such that when the garment is worn by the patient, the fabric defines two leg holes and an aperture, which is positioned adjacent to a body orifice of the patient so as to permit insertion of an endoscope through the aperture into the body orifice.

9. The garment according to claim 8, wherein the fabric comprises a sheet of material having front and rear portions joined by a central portion, which is cut to fit between the legs of the patient, and wherein the garment comprises further comprises one or more fasteners, which are adapted to fix the front and rear portions together so as to cover the pelvic area.

10. The garment according to claim 8, wherein the aperture in the garment is sized and located to permit insertion of the endoscope into an anus of the patient.

11. Apparatus for endoscopy, comprising:
an endoscope-adapted garment having an aperture sized and located to permit insertion of an endoscope therethrough into a body orifice of a patient while the garment is worn by the patient; and
an endoscope retaining device, which is fixed within the aperture so as to hold the endoscope in place when the endoscope is inserted into the body orifice.

12. The apparatus according to claim 11, wherein the endoscope retaining device comprises a rigid material having a bore therethrough, which is sized to fit snugly around the endoscope, and wherein the garment comprises a fastening assembly for fixing the retaining device within the aperture.

13. The apparatus according to claim 11, wherein the aperture in the garment is sized and located to permit insertion of the endoscope into an anus of the patient.

14. A method for endoscopic examination of a patient, comprising:
fitting a garment around a pelvic area of the patient, the garment having an aperture sized and located to permit insertion of an endoscope therethrough into a body orifice of a patient while the garment is worn by the patient; and
inserting an endoscope through the aperture into the body orifice.

15. The method according to claim 14, wherein the garment comprises a sheet of material, having front and rear portions joined by a central portion, which is cut to fit between the legs of the patient, and wherein fitting the garment comprises placing the central portion between the legs and fastening the front and rear portions together so as to cover the pelvic area.

16. The method according to claim 14, and comprising fixing a retaining device within the aperture, and wherein inserting the endoscope comprises inserting the endoscope through the retaining device so that the retaining device holds the endoscope in place when the endoscope is inserted into the body orifice.

17. The method according to claim 16, wherein the retaining device comprises a rigid material having a bore therethrough, which is sized to fit snugly around the endoscope, and wherein fixing the retaining device comprises fastening the retaining device to the garment.

18. The method according to claim 14, wherein the aperture in the garment is sized and located to permit insertion of the endoscope into an anus of the patient, and wherein inserting the endoscope comprises inserting the endoscope through the aperture into the anus.
